(19) United States
(12) Patent Application Publication
(10) Pub. No.: US 2022/0088239 A1
(43) Pub. Date: Mar. 24, 2022

(54) Germicidal UV Lamp Device

(71) Applicants: Timothy Baldwin, Utica, NY (US); John Yorke, Utica, NY (US)

(72) Inventors: Timothy Baldwin, Utica, NY (US); John Yorke, Utica, NY (US)

(21) Appl. No.: 17/387,530

(22) Filed: Jul. 28, 2021

Related U.S. Application Data
(60) Provisional application No. 63/035,476, filed on Jun. 5, 2020.

(51) Int. Cl. A61L 2/10

(52) U.S. Cl. CPC A61L 2/10; A61L 2202/11; A61L 2202/16; A61L 2202/20

(57) ABSTRACT

The disinfecting lamp device is intended to provide a simple, quick, and effective disinfecting device, that can inactivate pathogens like viruses and bacteria from frequently used surfaces. To accomplish this, the device includes a germicidal UV lamp device that can kill pathogens and disinfect any surface that the lamp illuminates. Further, the device is a portable, lightweight, hand-held, battery powered product that uses photonic energy of a specific nano-meter range of light, i.e., UV light source. The lamp device may be used quickly and effectively on surfaces, documents, keyboards, etc. for quick disinfecting. Furthermore, the product is designed to minimize and reduce any potential risk the operator may have in using the product by inherent safeties regarding UV light. Thus, the device is a safe, effective, non-chemical, and easy to use product to render viruses and bacterial pathogens inactive and non-reproducible.

The current application claims a priority to the U.S. Provisional Patent application Ser. No. 63/035,476 filed on Jun. 5, 2020. The U.S. provisional patent application 63/035,476 is revived within the two-month period for unintentional abandonment by Aug. 5, 2021.

### FIELD OF THE INVENTION

The present invention relates generally to a UV lamp device. More specifically, the present invention is an apparatus that can disinfect surfaces with the help of the UV lamp device.

### BACKGROUND OF THE INVENTION

The global pandemic situation is rapidly changing, and it is causing priorities to shift for a lot of us. Considering the current global crisis, it is advisable to modify our current technologies to contribute to the fight against the pandemic. Coronavirus is the name for a group of viruses, including MERS and SARS which infect mammals and cause respiratory illness ranging in severity. While we are currently facing the ongoing threat of the pandemic, it remains a fact that tens of thousands of Americans lose their lives to influenza annually as well. Thus, protecting patients, customers, workers, and our families is more important than ever before. To that end, disinfecting frequently used surfaces is extremely important, and an improved sterilization technique is needed to combat the spread of all viruses in the community. For decades, scientists have known about the disinfection ability of ultraviolet wavelengths, specifically germicidal UV (also known as UV-C). In recent years, germicidal UV helped stop the spread of numerous pathogens like the flu and the superbug.

It is an objective of the present invention to provide a simple, quick, and effective disinfecting device, that can inactivate pathogens like viruses and bacteria from frequently used surfaces. To accomplish this, the present invention comprises a germicidal UV lamp device that can kill pathogens and disinfect any surface that the lamp illuminates. More specifically, the present invention is a portable, lightweight, hand-held, battery powered product that uses photonic energy of a specific nano-meter range of light, i.e., UV light source, to provide germicidal, disinfecting, viricidal and bactericidal action on a number of pathogens, spores, bacteria and various microbes at an energy level sufficient to kill viruses. Accordingly, the lamp device may be used quickly and effectively on surfaces, documents, keyboards, etc. for quick disinfecting. Further, the product is designed to minimize and reduce any potential risk the operator may have in using the product by inherent safeties regarding UV light. Thus, the present invention is a safe, effective non-chemical, and easy to use product to render viruses and bacterial pathogens inactive and non-reproducible.

### BRIEF DESCRIPTION OF THE DRAWINGS

FIG. 1 is a block diagram of the present invention, wherein thinner flowlines represent electrical connections between components and thicker flowlines represent electronic connections between components.

FIG. 2 is a top-front-left perspective view of the present invention.

FIG. 3 is a bottom-rear-right perspective view of the present invention.

FIG. 4 is a front elevational view of the present invention.

FIG. 5 is a rear elevational view of the present invention.

FIG. 6 is a sectional view of the present invention taken along A-A′ of FIG. 5.

FIG. 7 is a top plan view of the present invention.

FIG. 8 is a bottom plan view of the present invention.

FIG. 9 is a right-side elevational view of the present invention.

### DETAIL DESCRIPTIONS OF THE INVENTION

All illustrations of the drawings are for the purpose of describing selected versions of the present invention and are not intended to limit the scope of the present invention.

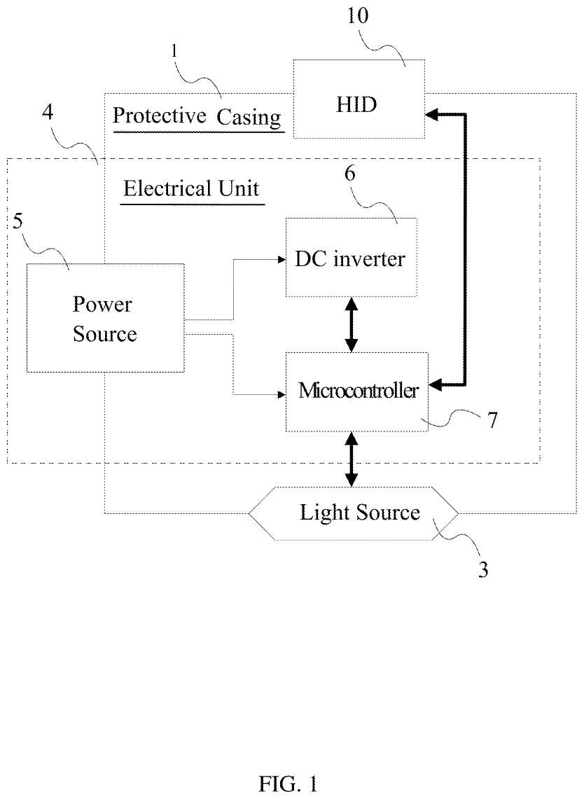

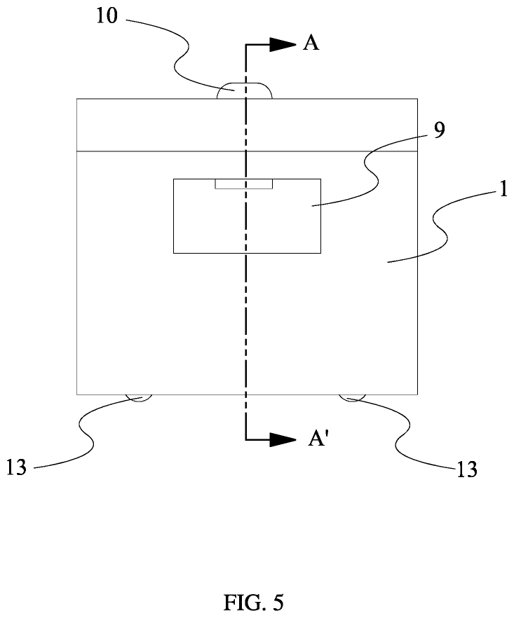

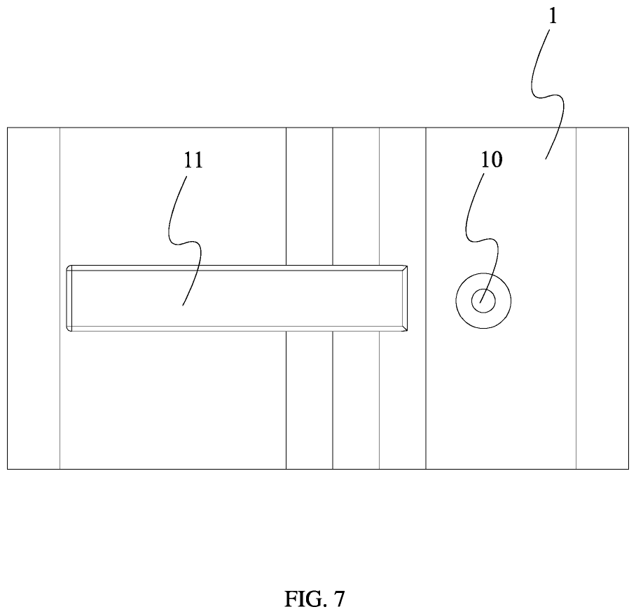

In reference to FIG. 1 through FIG. 9, the present invention is a disinfecting lamp device. It is an objective of the present invention to provide a simple, quick, and effective disinfecting device, that can inactivate pathogens like viruses and bacteria from frequently used surfaces. To accomplish this, the present invention comprises a germicidal UV lamp device that can kill pathogens and disinfect any surface that the lamp illuminates. More specifically, the present invention is a portable, lightweight, hand-held, battery powered product that uses photonic energy of a specific nano-meter range of light, i.e., UV light source, to provide germicidal, disinfecting, viricidal and bactericidal action on a number of pathogens, spores, bacteria and various microbes at an energy level sufficient to kill viruses. Accordingly, the lamp device may be used quickly and effectively on surfaces, documents, keyboards, etc. for quick disinfecting. Further, the product is designed to minimize and reduce any potential risk the operator may have in using the product by inherent safeties regarding UV light. Thus, the present invention is a safe, effective non-chemical, and easy to use product to render viruses and bacterial pathogens inactive and non-reproducible.

The following description is in reference to FIG. 1 through FIG. 9. According to a preferred embodiment, the present invention comprises a protective casing 1, an inner compartment 2, a germicidal light source 3, and an electrical unit 4. Preferably, the protective casing 1 comprises a rigid, sturdy, and light weight material, such as hard plastic, that can provide protection to the inner components housed within the protective casing. However, the protective casing 1 may comprise any other material, size, shape, components, arrangement of components etc. that are known to one of ordinary skill in the art, as long as the objectives of the present invention are fulfilled. Further, the protective casing 1 is designed to minimize and reduce any potential risk the operator may have in using the product that integrates UV light.

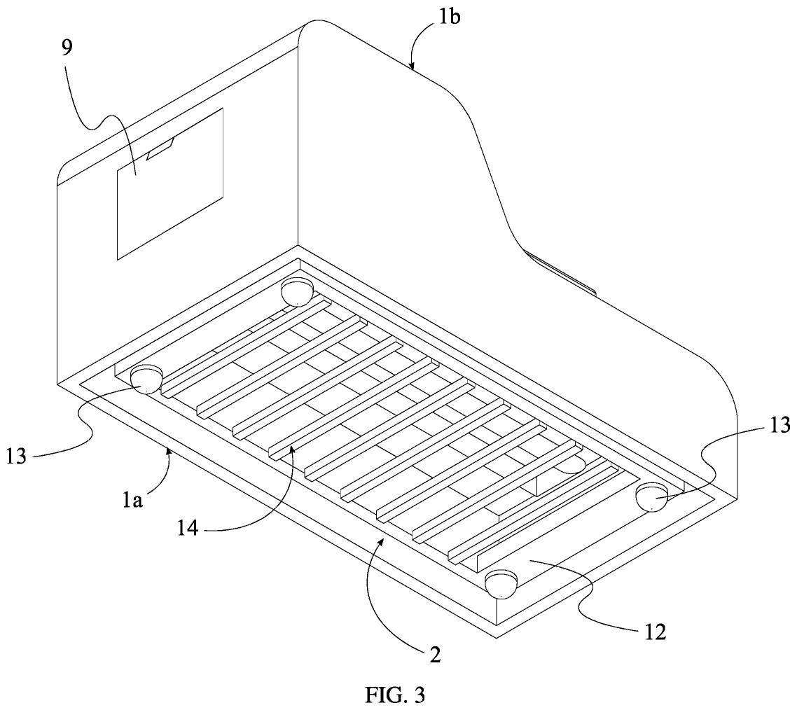

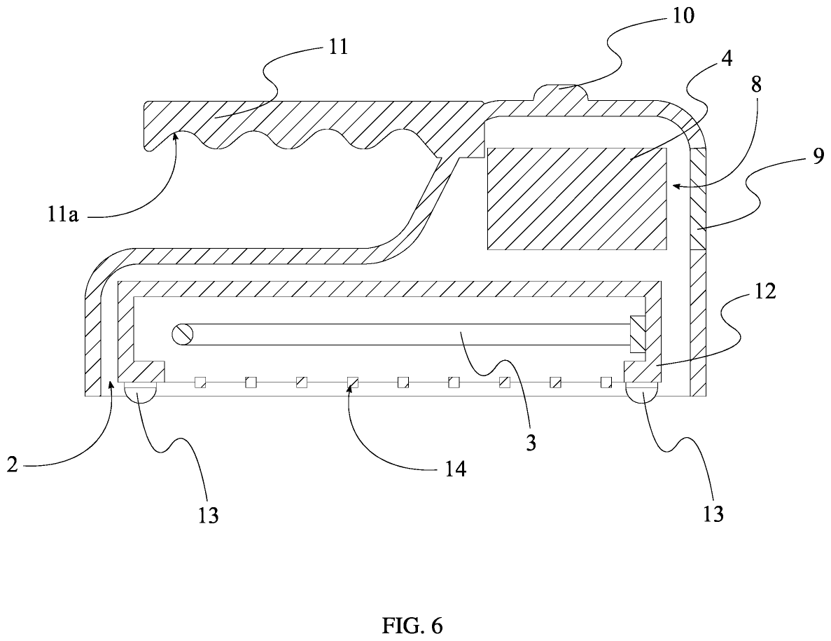

As seen in FIG. 3 and FIG. 6, the inner compartment 2 traverses into the protective casing 1. Preferably, the inner compartment 2 extends from a first end 1a towards a second end 1b, wherein the first end 1a is positioned opposite to the second end 1b across the protective casing 1. In other words, the inner compartment 2 is a hollow space that may comprise a variety of molded shapes and designs, such that the components of the present invention may fit, connect with each other, and function without any hindrance. Accordingly, the germicidal light source 3 and the electrical unit 4 are mounted within the inner compartment 2, such that the electrical unit 4 and the germicidal light source 3 are isolated from hazards in the external environment. In order to provide effective disinfection of preferred surfaces, the germicidal light source 3 is electrically connected to the electrical unit 4. Preferably, the germicidal light source 3 is a sealed glass tube bulb source, wherein the germicidal light source 3 energy provides germicidal, disinfecting, viricidal and bactericidal action on a number of pathogens, spores, bacteria and various microbes at an energy level sufficient to kill viruses. However, the germicidal light source 3 may comprise any other size, shape, technology etc. that are known to one of ordinary skill in the art, as long as the intents of the present invention are not altered. Examples of such sources include, but are not limited to sealed florescent tubes, UV bulbs, LED sources of a UV range and/or flash bulbs.

According to the preferred embodiment, the electrical unit 4 comprises a power source 5, a DC (direct current) inverter 6, and a microcontroller 7. This is because, the present invention employs a unique electronic technology that provides a battery cell configuration, wherein the DC inverter 6 is provided to allow the circuitry to create an artificial waveform which in turn allows the present invention to provide instant power to the germicidal light source 3 (AC bulb sufficient). In other words, the DC inverter 6 converts low DC power from a battery power source into adequate AC power, so as to energize the germicidal light source 3 to full energy deliverance capacity. Accordingly, it is preferred that the power source 5 is a 12 volt battery. Further, the power source 5 is also intended to deliver electrical power to the other components of the present invention. More specifically, the DC inverter 6 of the present invention has the ability to take a 9-12 volt input and convert it to the 45-55 volts, thereby providing sufficient input requirements for maximum operating efficiency to the germicidal light source 3 while controlling the amperage output so as to not damage the bulb filament resistor of the germicidal light source 3. To that end, the power source 5 is a rechargeable battery. However, any other source of power, or a combination of the following sources may be employed for the smooth functioning of the disinfecting lamp device. Examples of such power sources include, but are not limited to, Li ion batteries, magnetic power converters, solar power converters etc. The microcontroller 7 is a processing device that manages the operation of the electrical components within the present invention. However, the DC inverter 6, the power source 5 and the microcontroller 7 may comprise any size, brand, technology, etc. that are known to one of ordinary skill in the art, as long as the intents of the present invention are fulfilled. To accomplish the smooth functioning of the present invention, the power source 5, the DC inverter 6 and the microcontroller 7 are mounted within the electrical unit 4, such that the internal components are isolated from hazards in the external environment. As seen in FIG. 1, the power source 5 is electrically connected to the DC inverter 6, and the DC inverter 6 is electronically connected to the microcontroller 7.

Continuing with the preferred embodiment, the present invention comprises an access port 8 and an access door 9. As seen in FIG. 6, the access port 8 laterally traverses into protective casing 1, wherein the access port 8 extends into the electrical unit 4. Further, the access door 9 is operably integrated between the protective casing 1 and the access port 8, such that operating the access door 9 governs access to the power source 5 through the access port 8. Preferably, the access port 8 is a chamber for housing the power source 5, such as a rechargeable battery, and the access door 9 is a removably attachable door that leads to the battery chamber. However, the access port 8 and the access door 9 may comprise any other shape, size, fastening techniques, components, arrangement of components etc., that are known to one of ordinary skill in the art, as long as the intents of the present invention are not altered.

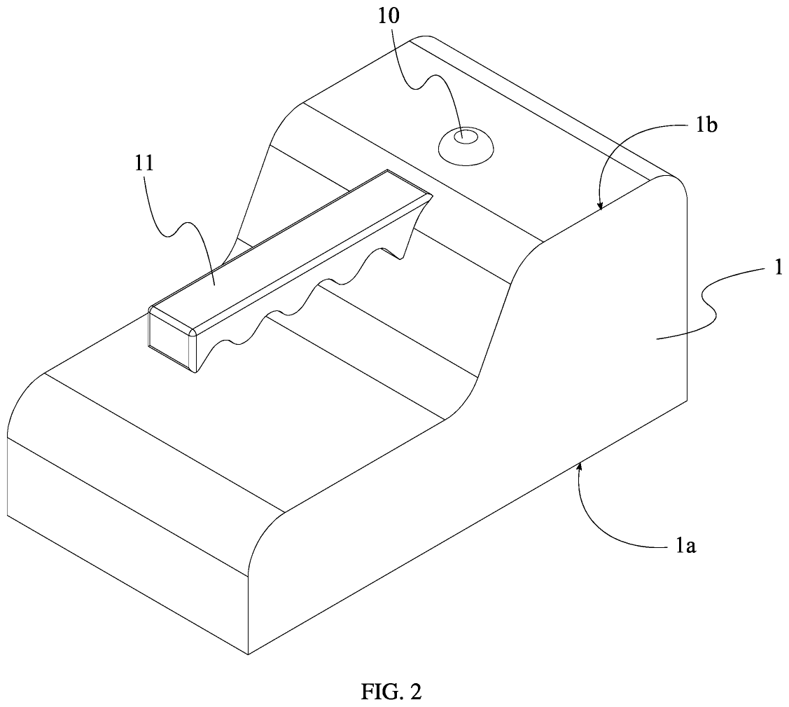

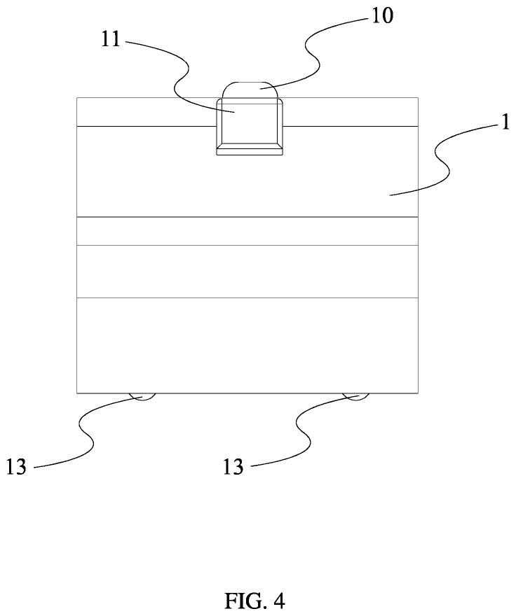

As seen in FIG. 1, FIG. 2, FIG. 4 through FIG. 7 and FIG. 9, the present invention comprises a human interface device (HID) 10. In the preferred embodiment, the HID 10 is integrated onto an external surface of the protective casing 1, and the HID 10 is electronically connected to the electrical unit 4. Preferably, the HID 10 forms the platform for user interaction in order to perform the various operations of the present invention. Accordingly, the HID 10 comprises one of more push-button switches that may perform various operations, including, but are not limited to, turning the device power on/off, and perform various other operations based on the capacities and modules on the microcontroller 7. In other words, the HID 10 is operatively coupled to the germicidal light source, wherein operating the HID 10 transitions the germicidal light source 3 between an ON state and an OFF state. Additionally, the HID 10 (on/off button) may become illuminated upon activation. The button will cease illumination during operation and 5 minutes before the battery capacity falls below the required output levels. The electrical unit 4 will always operate at full energy deliverance to the germicidal light source 3 sufficient to provide the germicidal light source 3 the ability to deliver a minimum of 100 mW/cm2 of the UV specific nm germicidal wavelength. The circuitry provides a constant voltage to the germicidal light source 3 which provides for this ability. As seen in FIG. 2, the HID 10 is positioned adjacent the second end 1b of the protective casing 1, for easy accessibility to the user. It should be noted that the HID 10 may comprise any other shape, size, orientation, location, components, arrangement of components etc., as long as the intents of the present invention are not altered.

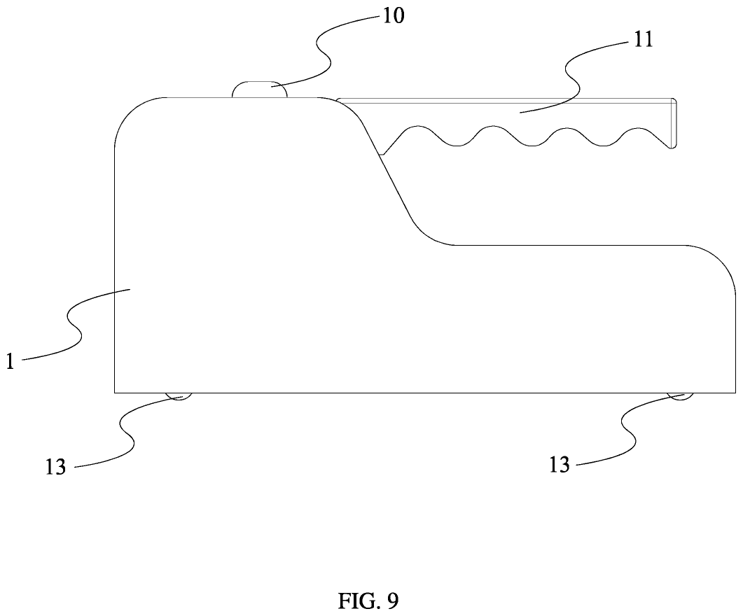

According to the preferred embodiment, the present invention is designed to be used on a horizontal plane, and the germicidal light source 3 is mounted horizontal to the surface intended to be treated. In other words, the germicidal light source 3 is mounted parallel to a horizontal plane of the protective casing 1. A top mounted handle allows for either right or left-handed utilization. Accordingly, in order to provide easy accessibility and handling of the present invention by the user, the present invention comprises a handle 11, wherein, the handle 11 is mounted adjacent the second end 1b of the protective casing 1. As seen in FIG. 2 and FIG. 6, FIG. 9, the handle 11 extends laterally from the protective casing 1 opposite the HID 10. Further, the handle 11 comprises an ergonomic grip 11a, for comfort and ease of handling.

In reference to FIG. 6, the present invention comprises an inner receptacle 12. Preferably, the inner receptacle 12 is mounted within the inner compartment 2 adjacent the first end 1a and the germicidal light source 3 is mounted within the inner receptacle 12. The inner receptacle 12 is intended to provide an additional casing and protective housing for the germicidal light source 3, and for separating the electrical components from the germicidal light source 3.

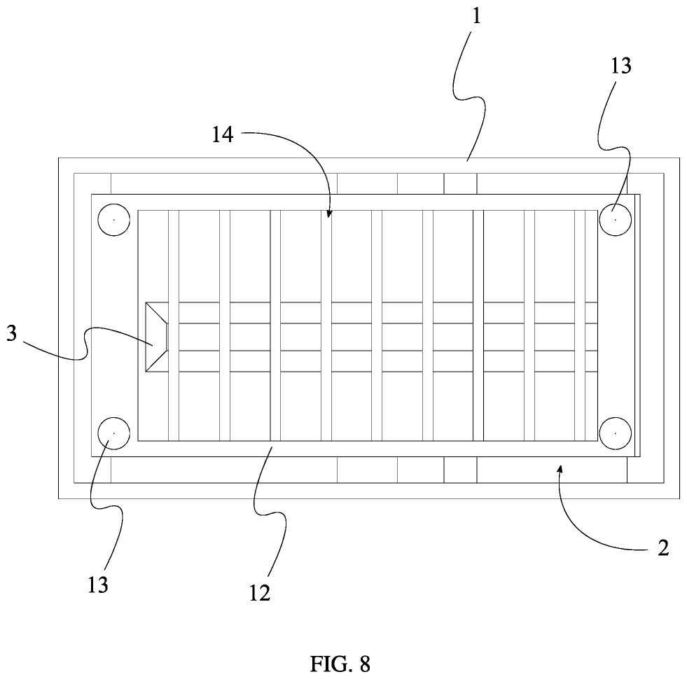

In order to provide support for the protective casing when being placed on a flat surface and preventing any scratching while using the device for disinfecting surfaces, the present invention comprises a plurality of support legs 13. Accordingly, the plurality of support legs 13 is perimetrically mounted adjacent the first end 1a of the protective casing 1, wherein the plurality of support legs 13 is oriented normally opposite to the second end 1b. As seen in FIG. 6 and FIG. 8, the plurality of support legs 1b is mounted terminally and perimetrically onto the inner receptacle 12. Preferably, the plurality of support legs 13 comprises bushes or rounded leg structures. However, the plurality of support legs 13 may comprise any other size, shape, location, number etc., as long as the intents of the present invention are fulfilled.

According to the preferred embodiment, the present invention comprises a protective panel 14. Preferably, the protective panel 14 is mounted adjacent the first end 1a of the protective casing 1, and the protective panel 14 covers the germicidal light source 3 in a protective fashion. It should be noted that the protective panel 14 enables light from the germicidal light source 3 to come out through the first end 1a. As seen in FIG. 3 and FIG. 8, the protective panel 14 is a grill structure. However, the protective panel 14 may comprise any other shape, structure, components, arrangement of components etc., that are known to one of ordinary skill in the art, as long as the intents of the present invention are fulfilled. Examples of such panels include, but are not limited to, transparent plastic panel, glass panel, grid structure etc.

Although the invention has been explained in relation to its preferred embodiment, it is to be understood that many other possible modifications and variations can be made without departing from the spirit and scope of the invention as hereinafter claimed.

What is claimed is:

1. A disinfecting lamp device comprising:
a protective casing;
an inner compartment;
a germicidal light source;
an electrical unit;
the inner compartment traversing into the protective casing;
the inner compartment extending from a first end of the protective casing towards a second end of the protective casing, wherein the first end is positioned opposite to the second end across the protective casing;
the germicidal light source and the electrical unit being mounted within the inner compartment;
the germicidal light source being electrically connected to the electrical unit.

2. The disinfecting lamp device of claim 1, comprising:
a power source;
a DC (direct current) inverter;
a microcontroller;
the power source, the DC inverter and the microcontroller being mounted within the electrical unit;
the power source being electrically connected to the DC inverter; and
the DC inverter being electronically connected to the microcontroller.

3. The disinfecting lamp device of claim 2, comprising:
an access port;
an access door;
the access port laterally traversing into protective casing;
the access port extending into the electrical unit;
the access door being operably integrated between the protective casing and the access port, wherein operating the access door governs access to the power source through the access port.

4. The disinfecting lamp device of claim 1, comprising:
a human interface device (HID);
the HID being integrated onto an external surface of the protective casing;
the HID being electronically connected to the electrical unit;
the HID being operatively coupled to the germicidal light source, wherein operating the HID transitions the germicidal light source between an ON state and an OFF state.

5. The disinfecting lamp device of claim 4, wherein the HID being positioned adjacent the second end of the protective casing.

6. The disinfecting lamp device of claim 4, wherein the HID being a push button.

7. The disinfecting lamp device of claim 1, comprising:
a handle;
the handle being mounted adjacent the second end of the protective casing;
the handle extending laterally from the protective casing opposite the HID.

8. The disinfecting lamp device of claim 7, wherein the handle comprising an ergonomic grip.

9. The disinfecting lamp device of claim 1, comprising:
an inner receptacle;
the inner receptacle being mounted within the inner compartment adjacent the first end; and
the germicidal light source being mounted within the inner receptacle.

10. The disinfecting lamp device of claim 9, comprising:
a plurality of support legs;
the plurality of support legs being perimetrically mounted adjacent the first end of the protective casing;
the plurality of support legs being oriented normally opposite to the second end.

11. The disinfecting lamp device of claim 9, wherein the plurality of support legs being mounted terminally and perimetrically onto the inner receptacle.

12. The disinfecting lamp device of claim 1, comprising:
a protective panel;
the protective panel being mounted adjacent the first end of the protective casing;
the protective panel covering the germicidal light source; and
the protective panel enabling light from the germicidal light source to traverse through the first end.

13. The disinfecting lamp device of claim 12, wherein the protective panel being a grill structure.

14. A disinfecting lamp device comprising:
a protective casing;
an inner compartment;
a germicidal light source;
an electrical unit;
the electrical unit comprising a power source, a DC (direct current) inverter, and a microcontroller;
the inner compartment traversing into the protective casing;
the inner compartment extending from a first end towards a second end, wherein the first end is positioned opposite to the second end across the protective casing;
the germicidal light source and the electrical unit being mounted within the inner compartment;
the germicidal light source being electrically connected to the electrical unit;
the power source, the DC inverter and the microcontroller being mounted within the electrical unit;
the power source being electrically connected to the DC inverter; and
the DC inverter being electronically connected to the microcontroller.

15. The disinfecting lamp device of claim 14, comprising:
a human interface device (HID);
the HID being integrated onto an external surface of the protective casing;
the HID being positioned adjacent the second end of the protective casing;
the HID being electronically connected to the electrical unit; and
the HID being operatively coupled to the germicidal light source, wherein operating the HID transitions the germicidal light source between an ON state and an OFF state.

16. The disinfecting lamp device of claim 14, comprising:
a handle;
the handle being mounted adjacent the second end of the protective casing; and
the handle extending laterally from the protective casing opposite the HID.

17. The disinfecting lamp device of claim 14, comprising:
an inner receptacle;
the inner receptacle being mounted within the inner compartment adjacent the first end; and
the germicidal light source being mounted within the inner receptacle.

18. The disinfecting lamp device of claim 17, comprising:
a plurality of support legs;
the plurality of support legs being perimetrically mounted adjacent the first end of the protective casing;
the plurality of support legs being mounted terminally and perimetrically onto the inner receptacle; and
the plurality of support legs being oriented normally opposite to the second end.

19. The disinfecting lamp device of claim 14, comprising:
a protective panel;
the protective panel being mounted adjacent the first end of the protective casing;
the protective panel covering the germicidal light source; and
the protective panel enabling light from the germicidal light source to come out through the first end.
